(19) United States
(12) Patent Application Publication
(10) Pub. No.: US 2025/0218574 A1
(43) Pub. Date: Jul. 3, 2025

(54) SYSTEMS AND METHODS FOR IMAGE SEQUENCE REGISTRATION

(71) Applicant: Stryker Corporation, Portage, MI (US)

(72) Inventors: Xudong LU, West Vancouver (CA); Ian SCOTT, North Vancouver (CA)

(73) Assignee: Stryker Corporation, Portage, MI (US)

(21) Appl. No.: 19/002,403

(22) Filed: Dec. 26, 2024

Related U.S. Application Data
(60) Provisional application No. 63/615,751, filed on Dec. 28, 2023.

(51) Int. Cl. G16H 30/40; G06T 7/38

(52) U.S. Cl. CPC G16H 30/40; G06T 7/38; G06T 2207/10016

(57) ABSTRACT

A method of registering frames of a medical video includes, at a computing system, receiving a series of video frames captured by a medical imager imaging a scene of interest of a patient, for a first frame of the series of video frames, determining a plurality of registration estimates for registering the first frame to a second frame of the series of video frames, wherein each registration estimate is determined according to a different one of a plurality of registration methods, determining registration accuracies for the plurality of registration estimates, selecting one of the plurality of registration estimates based on the registration accuracies, and storing in a memory the selected registration estimate as a registration of the first frame to the second frame.

### CROSS-REFERENCE TO RELATED APPLICATION

This application claims the benefit of U.S. Provisional Application No. 63/615,751, filed on Dec. 28, 2023, the entire content of which is incorporated herein by reference for all purposes.

### FIELD

This disclosure relates generally to medical imaging, and more specifically, to medical image sequence registration.

### BACKGROUND

Video registration involves the registering of sequential image frames to a selected reference frame. This is often a challenging task in medical imaging, particularly handheld medical imaging, because the field of view in the video can change significantly over time. The change in the field of view can result from intentional movement of the camera by the user to improve visualization, irregular camera motion caused by hand shaking or other inadvertent movement, or other intentional or unintentional movement of the camera. Additionally, the imaging target (e.g., the tissue under observation) may deform and/or move relative to the camera.

Image registration is a well-known task. It matches local or global salient features from an image pair and estimates a transform (e.g., translation, affine, perspective, etc.). Many approaches used to solve registration for sequential video frames are an extension of image pair registration. Based on a selected reference frame, subsequent frames can be registered to the selected reference frame. However, frames captured later in time with large time gaps with the reference frame are typically harder to register to the reference frame due to changes in the field of view from camera movements or deformation of the target.

### SUMMARY

According to an aspect, systems and methods described herein can register video frames to a reference frame by determining registration estimates using a plurality of different registration methods and selecting the registration estimate that provides a better registration accuracy. For a frame of interest of a series of video frames, multiple registration estimates are determined using different registration methods for registering the frame of interest to a reference frame. The registration methods can include, for example, calculating a transform directly between the frame of interest and the reference frame, accumulating transforms between adjacent frames leading from the frame of interest back in time to the reference frame to compute a global transform from the reference frame to the frame of interest, and calculating a transform between the frame of interest and a key frame and accumulating that transform with a transform from the key frame to the reference frame. Registration accuracies may be determined for the various registration estimates and a registration estimate with a better accuracy may be selected as the registration for the frame of interest. The different registration methods may each have their advantages and disadvantages relative to the other registration method(s). By comparing registration estimates determined using different methods and selecting the one that has the best accuracy for the given frame of interest, the registration process can adapt to each new frame of interest.

A registration estimate can be determined using a key frame by selecting a key frame from a set of key frames based on a similarity between the frame of interest and the selected key frame. The similarity between the frame of interest and the key frames can be determined based on the spatial locations of their scenes relative to the scene of the reference frame. For example, a location of a center of the frame of interest transformed using a registration estimate determined by a registration method that does not use the key frames can be compared to locations of the key frames relative to the reference frame and the key frame that is sufficiently close may be selected. The set of key frames can be logically organized in a two-dimensional grid whose cells define locations relative to the reference frame. Each cell can be assigned a key frame based on the location of the key frame corresponding to the cell. The cell corresponding to the location of the frame of interest is determined and its assigned key frame is selected. The key frame database can be built over time by assigning frames to the database when, for example, no key frame has been assigned to a given cell and/or by replacing key frames when a frame of interest has a registration accuracy that exceeds the registration accuracy of the key frame.

According to an aspect, a method of registering frames of a medical video includes, at a computing system: receiving a series of video frames captured by a medical imager imaging a scene of interest of a patient; for a first frame of the series of video frames, determining a plurality of registration estimates for registering the first frame to a second frame of the series of video frames, wherein each registration estimate is determined according to a different one of a plurality of registration methods; determining registration accuracies for the plurality of registration estimates; selecting one of the plurality of registration estimates based on the registration accuracies; and storing in a memory the selected registration estimate as a registration of the first frame to the second frame.

The series of video frames may include at least one frame that was captured between the first frame and the second frame, and the plurality of registration methods may include at least one registration method that includes calculating a transform from the first frame directly to the second frame. The plurality of registration methods may include at least one registration method that comprises calculating a transform from the first frame to an intermediate frame that was captured between the first and second frames. The at least one registration method may include combining the transform from the first frame to the intermediate frame with a plurality of transforms associated with frames captured between the intermediate frame and the second frame. The intermediate frame may be a key frame and the at least one registration method may include combining the transform from the first frame to the key frame with a second transform from the key frame to the second frame. The key frame may be a selected key frame that was selected from a plurality of key frames based on a similarity between the first frame and the key frame. The at least one registration method that comprises combining the transform from the first frame to the selected key frame with the second transform may be a first registration method and the selected key frame may be selected based on a second registration method. The second registration method may include combining a plurality of transforms associated with frames captured between the first frame and the second frame. The method may include determining a relative difference in location between the first frame and the second frame, wherein the selected key frame is selected based on the relative difference in location between the first frame and the second frame. The relative difference in location between the first frame and the second frame may include a relative difference in location between a center of the first frame and a center of the second frame. The selected key frame may be selected by determining which key frame of the plurality of key frames has a location corresponding to the first frame. Each key frame of the plurality of key frames may be associated with a range of locations relative to the second frame and the selected key frame is selected by determining that the relative difference in location between the first frame and the second frame is within a range associated with the selected key frame. The plurality of key frames may be frames of the series of video frames.

Optionally, the method includes determining a location of the first frame relative to the second frame based on a first registration estimate; determining whether a key frame database includes a key frame that is sufficiently close in location to the first frame; in accordance with the key frame database not including a key frame that is sufficiently close in location to the first frame, adding the first frame to the key frame database; and in accordance with the key frame database including a key frame that is sufficiently close in location to the first frame: comparing a registration accuracy associated with the key frame and a registration accuracy associated with the first registration estimate for the first frame, and replacing the key frame with the first frame if the registration accuracy associated with the first registration estimate is greater than the registration accuracy associated with the key frame.

Selecting the one of the plurality of registration estimates based on the registration accuracies may include comparing a first registration accuracy corresponding to a first registration estimate to a threshold, and in accordance with the first registration accuracy not meeting the threshold, comparing the first registration accuracy to a second registration accuracy corresponding to a second registration estimate. At least one registration method of the plurality of registration methods may include determining at least one homography estimate.

The at least one homography estimate may be determined using a random sample consensus algorithm. The at least one homography estimate may be determined using a machine learning-based algorithm. The medical imager may be an endoscopic imager.

The method may include registering pixel data of the first frame to pixel data of the second frame based on the selected registration estimate. The series of video frames may be associated with a first imaging modality, and the method may include registering a frame of a series of video frames associated with a second imaging modality based on the selected registration estimate.

According to an aspect, a system for registering frames of a medical video includes one or more processors and memory storing one or more programs for execution by the one or more processors, the one or more programs including instructions that when executed by the one or more processors cause the system to perform a method that includes: receiving a series of video frames captured by a medical imager imaging a scene of interest of a patient; for a first frame of the series of video frames, determining a plurality of registration estimates for registering the first frame to a second frame of the series of video frames, wherein each registration estimate is determined according to a different one of a plurality of registration methods; determining registration accuracies for the plurality of registration estimates; selecting one of the plurality of registration estimates based on the registration accuracies; and storing in the memory the selected registration estimate as a registration of the first frame to the second frame. Optionally, the system includes the medical imager. The medical imager may be, for example, an endoscopic imager.

The series of video frames may include at least one frame that was captured between the first frame and the second frame, and the plurality of registration methods may include at least one registration method that includes calculating a transform from the first frame directly to the second frame. The plurality of registration methods may include at least one registration method that comprises calculating a transform from the first frame to an intermediate frame that was captured between the first and second frames. The at least one registration method may include combining the transform from the first frame to the intermediate frame with a plurality of transforms associated with frames captured between the intermediate frame and the second frame. The intermediate frame may be a key frame and the at least one registration method may include combining the transform from the first frame to the key frame with a second transform from the key frame to the second frame. The key frame may be a selected key frame that was selected from a plurality of key frames based on a similarity between the first frame and the key frame. The at least one registration method that comprises combining the transform from the first frame to the selected key frame with the second transform is a first registration method and the selected key frame may be selected based on a second registration method. The second registration method may include combining a plurality of transforms associated with frames captured between the first frame and the second frame. The one or more programs may include instructions for determining a relative difference in location between the first frame and the second frame, wherein the selected key frame is selected based on the relative difference in location between the first frame and the second frame. The relative difference in location between the first frame and the second frame may include a relative difference in location between a center of the first frame and a center of the second frame. The selected key frame may be selected by determining which key frame of the plurality of key frames has a location corresponding to the first frame. Each key frame of the plurality of key frames may be associated with a range of locations relative to the second frame and the selected key frame may be selected by determining that the relative difference in location between the first frame and the second frame is within a range associated with the selected key frame. The plurality of key frames may be frames of the series of video frames.

The one or more programs may include instructions for: determining a location of the first frame relative to the second frame based on a first registration estimate; determining whether a key frame database includes a key frame that is sufficiently close in location to the first frame; in accordance with the key frame database not including a key frame that is sufficiently close in location to the first frame, adding the first frame to the key frame database; and in accordance with the key frame database including a key frame that is sufficiently close in location to the first frame: comparing a registration accuracy associated with the key frame and a registration accuracy associated with the first registration estimate for the first frame, and replacing the key frame with the first frame if the registration accuracy associated with the first registration estimate is greater than the registration accuracy associated with the key frame.

Selecting the one of the plurality of registration estimates based on the registration accuracies may include comparing a first registration accuracy corresponding to a first registration estimate to a threshold, and in accordance with the first registration accuracy not meeting the threshold, comparing the first registration accuracy to a second registration accuracy corresponding to a second registration estimate.

Optionally, at least one registration method of the plurality of registration methods comprises determining at least one homography estimate. The at least one homography estimate may be determined using a random sample consensus algorithm. The at least one homography estimate may be determined using a machine learning-based algorithm.

The one or more programs may include instructions for registering pixel data of the first frame to pixel data of the second frame based on the selected registration estimate. The series of video frames may be associated with a first imaging modality, and the method may include registering a frame of a series of video frames associated with a second imaging modality based on the selected registration estimate.

According to an aspect, a non-transitory computer readable storage medium stores one or more programs for execution by one or more processors of a computing system, the one or more programs including instructions for causing the computing system to perform any of the methods above.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an exemplary imaging system for imaging tissue of a subject;

FIG. 2 is a flow diagram of an exemplary method for registering frames of a series of video frames;

FIG. 3 illustrates examples of different registration methods;

FIG. 4 is a flow diagram of an exemplary method for determining a registration estimate using a plurality of different registration methods;

FIG. 5 is a flow diagram of an exemplary method for determining which key frame from among a set of key frames to select for registering a frame of interest;

FIG. 6 illustrates an example of determining the location of a frame of interest relative to a reference frame for selecting a key frame;

FIG. 7 illustrates an example of using registration of a series of first imaging modality frames for registering a series of second imaging modality frames; and

FIG. 8 is a functional block diagram of an exemplary computing system.

### DETAILED DESCRIPTION

In the following description of the various examples, reference is made to the accompanying drawings, in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art and the generic principles herein may be applied to other examples. Thus, the present invention is not intended to be limited to the examples shown but is to be accorded the widest scope consistent with the principles and features described herein.

According to an aspect, systems and methods described herein can register video frames to a reference frame by determining registration estimates using a plurality of different registration methods and selecting the registration estimate that provides a better registration accuracy. The frame registration according to the principals described herein can utilize accumulated adjacent registration with global regularization to avoid divergency through key frame matching as a check on the accumulated adjacent registration. Furthermore, an efficient multiple key frames management approach facilitates the searching and matching processes to avoid increasing additional registration latency.

Different registration methods may be used in sequential fashion, such as based on the failure of a previous registration method to produce an adequate registration estimate. Alternatively, multiple registration methods may be used to generate multiple registration estimates and the accuracies of those registration estimates may then be compared to determine which to use.

According to some variations, a registration process for a given frame (a frame of interest) in a sequence of video frames can include determining multiple different registration estimates using different registration methods. In one registration method, a registration estimate is determined by accumulating transforms (e.g., homographic transforms) between adjacent frames in the sequence to provide a global registration of the frame of interest to the reference frame. An accuracy for the registration estimate can be determined. If the accuracy is below a threshold, then one or more other registration methods may be used to determine one or more other registration estimates.

Another registration method that may be used to determine a registration estimate is determining a transform that can warp the frame of interest directly to the reference frame. The accuracy of a registration estimate produced by the registration method can be compared to a threshold (the same threshold as used for the first method or a different threshold). If it also fails to meet the threshold, then yet another registration method may be used. A third registration method that may be used may register the frame of interest to the most similar key frame from a key frame database and obtain the overall registration to the reference frame by accumulating the transforms from the frame of interest to the key frame and from the key frame to the reference frame.

Efficient key frame management can enable registration using key frames without excessive computation costs (i.e., with low latency). A set of key frames can be organized as a two-dimensional grid to which key frames are assigned based on their locations relative to the reference frame. A location of a frame of interest can be determined using a registration estimate determined without using the key frames. The cell of the grid that corresponds to the location of the frame of interest can be used to determine the key frame that is most similar to the frame of interest. If a cell does not have a key frame assigned to it, the frame of interest can be assigned to the cell as a key frame. If the cell has an assigned key frame, a comparison may be made between the frame of reference and the key frame to see which one is more similar to the reference frame by a suitable similarity measurement. The one with the highest similarity may be assigned to the cell.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

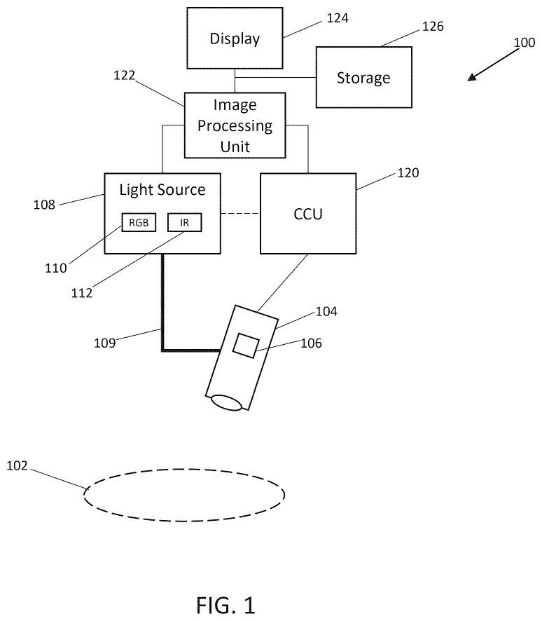

FIG. 1 illustrates an exemplary imaging system 100 for imaging tissue 102 of a subject during an imaging session, which can be an imaging session occurring during a surgical procedure or a non-surgical medical procedure. The system 100 includes a medical imager 104 that has at least one image sensor 106 configured to capture images (e.g., single snapshot images and/or a series of video frames) depicting the tissue and/or one or more features of the tissue. (The terms image and frame are used interchangeably throughout.) The imager 104 can be a hand-held device, such as an open-field camera or an endoscopic camera or can be mounted or attached to a mechanical support arm.

The imager 104 may be connected to a camera control unit (CCU) 120, which may generate one or more single snapshot images and/or video frames (referred to herein collectively as images) from imaging data generated by the imager 104. The images generated by the camera control unit 120 may be transmitted to an image processing unit 122 that may apply one or more image processing techniques described further below to the images generated by the camera control unit 120. Optionally, the camera control unit 120 and the image processing unit 122 are integrated into a single device. The image processing unit 122 (and/or the camera control unit 120) may be connected to one or more displays 124 for displaying the one or more images generated by the camera control unit 120 or one or more images or other visualizations generated based on the images generated by the camera control unit 120. The image processing unit 122 (and/or the camera control unit 120) may store the one or more images generated by the camera control unit 120 or one or more images or other visualizations generated based on the images generated by the camera control unit 120 in one or more storage devices 126. The one or more storage devices can include one or more local memories, one or more remote memories, a recorder or other data storage device, a printer, and/or a picture archiving and communication system (PACS). The system 100 may additionally or alternatively include any suitable systems for communicating and/or storing images and image-related data.

The imaging system 100 may include a light source 108 configured to generate light that is directed to the field of view to illuminate the tissue 102. Light generated by the light source 108 can be provided to the imager 104 by a light cable 109. The imager 104 may include one or more optical components, such as one or more lenses, fiber optics, light pipes, etc., for directing the light received from the light source 108 to the tissue. The imager 104 may be an endoscopic camera that includes an endoscope that includes one or more optical components for conveying the light to a scene within a surgical cavity into which the endoscope is inserted. The imager 104 may be an open-field imager and may include one or more lenses that direct the light toward the field of view of the open-field imager.

The light source 108 includes one or more visible light emitters 110 that emit visible light in one or more visible wavebands (e.g., full spectrum visible light, narrow band visible light, or other portions of the visible light spectrum). The visible light emitters 110 may include one or more solid state emitters, such as LEDs and/or laser diodes. The visible light emitters 110 may include red, green, and blue (or other color component) LEDs or laser diodes that in combination generate white light or other illumination needed for reflected light imaging. These color component light emitters may be centered around the same wavelengths around which the imager 104 is centered. For example, in variations in which the imager 104 includes a single chip, single color image sensor having an RGB color filter array deposited on its pixels, the red, green, and blue light sources may be centered around the same wavelengths around which the RGB color filter array is centered. As another example, in variations in which the imager 104 includes a three-chip, three-sensor (RGB) color camera system, the red, green, and blue light sources may be centered around the same wavelengths around which the red, green, and blue image sensors are centered.

The light source 108 can include one or more excitation light emitters 112 configured to emit excitation light suitable for exciting intrinsic fluorophores and/or extrinsic fluorophores (e.g., a fluorescence imaging agent that has been introduced into the subject) located in the tissue being imaged. The excitation light emitters 112 may include, for example, one or more LEDs, laser diodes, arc lamps, and/or illuminating technologies of sufficient intensity and appropriate wavelength to excite the fluorophores located in the object being imaged. For example, the excitation light emitter(s) may be configured to emit light in the near-infrared (NIR) waveband (such as, for example, approximately 805 nm light), though other excitation light wavelengths may be appropriate depending on the application.

The light source 108 may further include one or more optical elements that shape and/or guide the light output from the visible light emitters 110 and/or excitation light emitters 112. The optical components may include one or more lenses, mirrors (e.g., dichroic mirrors), light guides and/or diffractive elements, e.g., to help ensure a flat field over substantially the entire field of view of the imager 104.

The imager 104 may acquire reflected light images from visible light reflected from the tissue that is incident on the at least one image sensor 106 and/or fluorescence images from fluorescence light emitted by fluorophores in the tissue (which are excited by the fluorescence excitation light) that is incident on the at least one image sensor 106. The at least one image sensor 106 may include at least one solid state image sensor. The at least one image sensor 106 may include, for example, a charge coupled device (CCD), a CMOS sensor, a CID, or other suitable sensor technology. The at least one image sensor 106 may include a single image sensor (e.g., a grayscale image sensor or a color image sensor having an RGB color filter array deposited on its pixels). The at least one image sensor 106 may include multiple sensors, such as one sensor for detecting red light, one for detecting green light, and one for detecting blue light.

The camera control unit 120 can control timing of image acquisition by the imager 104. The imager 104 may be used to acquire both reflected light images and fluorescence images and the camera control unit 120 may control a timing scheme for the imager 104. The camera control unit 120 may be connected to the light source 108 for providing timing commands to the light source 108. Alternatively, the image processing unit 122 may control a timing scheme of the imager 104, the light source 108, or both.

The timing scheme of the imager 104 and the light source 108 may enable capture of reflected light images and fluorescence light images in an alternating fashion. In particular, the timing scheme may involve illuminating the tissue with illumination light and/or excitation light according to a pulsing scheme and processing the reflected light image and fluorescence image with a processing scheme that is synchronized and matched to the pulsing scheme to enable separation of the two types of images in a time-division multiplexed manner. Examples of such pulsing and image processing schemes have been described in U.S. Pat. No. 9,173,554, filed on Mar. 18, 2009, and titled “IMAGING SYSTEM FOR COMBINED FULL-COLOR REFLECTANCE AND NEAR-INFRARED IMAGING,” the contents of which are incorporated in their entirety by this reference. However, other suitable pulsing and image processing schemes may be used to acquire reflected light video frames and fluorescence video frames.

System 100 may be configured to generate and process reflected light images and fluorescence light images to analyze spatial and temporal characteristics of blood flow in the tissue 102. For example, the image processing unit 122 may analyze characteristics of brightness within fluoresce light frames of a series of video frames to quantify blood flow in the tissue 102. The analysis may include analyzing brightness from multiple fluorescence light frames in the series, such as to track changes in the brightness over time. Since the tissue 102 and the imager 104 may move relative to one another, the image processing unit 122 may register frames to correct for such movement. Registration of the fluorescence light frames may be performed based on registration of reflected light frames of the series of video frames. Using the reflected light frames to register the fluorescence light frames can provide more accurate registration since the reflected light frames typically contain more spatial information than the fluorescence light frames.

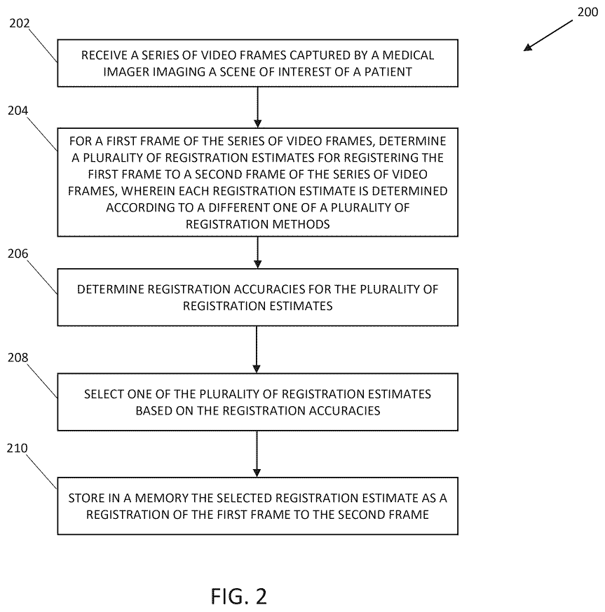

FIG. 2 is a flow diagram of an exemplary method 200 for registering frames of a series of video frames. Method 200 may be performed to register reflected light frames of a series of video frames. The registration performed by method 200 can be used to register fluorescence light frames that are captured close in time to the reflected light frames, such as when reflected light frames and fluorescence light frames are captured in alternating fashion, as discussed above. However, the registration of frames resulting from method 200 is not limited to use in registering fluorescence light frames. Other uses include motion deblurring, virtual marker placement and visualization, and navigation. Method 200 can be performed by a suitable computing system that can access a series of video frames. For example, method 200 can be performed by image processing unit 122 of system 100 based on video frames captured by imager 104.

Step 202 of method 200 includes receiving a series of video frames captured by a medical imager imaging a scene of interest of a patient. The series of video frames may have been captured by, for example, imager 104 of system 100. The video frames may have been captured by any type of imager, including, for example, an endoscopic camera, an open-field camera, a microscopic camera, or any other type of camera. The imager (e.g., endoscopic camera) may have been pre-inserted into the patient prior to performance of step 202. The video frames may have been captured during a medical procedure on the patient, which can be a surgical procedure or a non-surgical procedure.

The series of video frames can include reflected light frames, such as white light or other visible light frames, captured by an imager from light reflected from a scene of interest. The series of video frames may include just the reflected light frames or may include fluorescence light frames, which may have been captured in alternating fashion with the reflected light frames. The scene of interest can include, for example, tissue 102 of FIG. 1. The series of video frames may be received at a computing system, such as image processing unit 122 of system 100. The series of video frames may be received contemporaneously with when they are generated (e.g., in real-time) or may be received at some point after they are generated. For example, the video frames may be stored for later access by the computing system, and step 202 may include the computing system receiving the video frames from the storage.

At step 204, for a frame of interest of the series of video frames, multiple registration estimates are determined for registering the frame of interest to a reference frame of the series of video frames. For example, where a series of video frames includes frame 0 to frame N and frame 0 is a reference frame, multiple registration estimates are determined for registering frame N to frame 0. The registration estimates each estimate the motion that occurred between frames and, thus, estimate how pixels in the frame of interest relate to pixels in the reference frame.

The reference frame can be any frame in the series of video frames. For example, the reference frame can be the first frame in the series of video frames or can be a frame designated based on the occurrence of an event, such as the start of an analysis mode that utilizes frame registration, which may be initiated in response to a user command. The frame of interest can be any frame captured after the reference frame. The frame of interest can be, for example, a current frame, which can be a most-recently captured frame. The frame of interest can be a most-recently captured frame of a predefined proportion of the frames. For example, the imager may capture frames at 60 frames per second and method 200 may be performed at 30 frames per second such that step 204 may be performed on the most-recent frame at 30 frames per second.

The plurality of registration estimates determined at step 204 are determined using different registration methods. As described further below, the accuracies of the different registration estimates are determined and compared, and one of the registration estimates is selected based on the determined accuracies as the registration of the frame of interest to the reference frame.

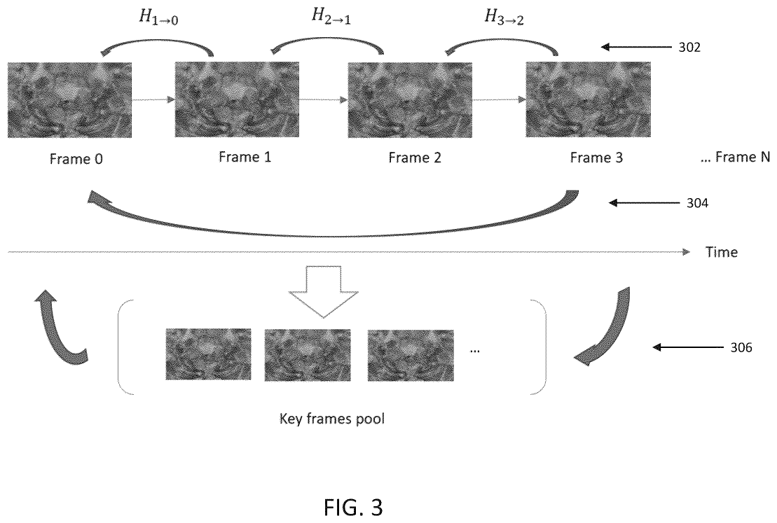

FIG. 3 illustrates examples of different registration methods that may be used to determine the registration estimates according to step 204. In the illustrated example, the series of video frames includes frame 0 to frame 3 captured over time that progresses from left to right (e.g., frame 1 was captured after frame 0). Frame 0 represents the reference frame to which later frames are registered according to method 200. Frame 3 is a frame of interest. The registration methods each find a reprojection transform to warp the frame of interest to the reference frame.

A first registration method 302 that may be used to determine a registration estimate for registering the frame of interest to a reference frame computes a transform between each set of adjacent frames (adjacent in time) and combines the transforms, resulting in a registration estimate from frame N to frame 0. In the illustrated example, a homographic transform H is computed for each set of adjacent frames, resulting in a transform from frame 3 to frame 2 (H_{3→2}), from frame 2 to frame 1 (H_{2→1}), and from frame 1 to frame 0 (H_{1→0}). The three homographic transforms are accumulated (e.g., multiplied), providing a homographic transform from frame 3 to frame 0 as follows:

$${H}_{3 \to 0} = {H}_{1 \to 0} * {H}_{2 \to 1} * {H}_{3 \to 2}$$

A second registration method 304 that may be used to determine a registration estimate for registering the frame of interest to a reference frame computes a transform between the frame of interest to the reference frame. In the illustrated example, a homographic transform is computed from frame 3 to frame 0 (H_{3→0}).

A third registration method 306 that may be used to determine a registration estimate for registering the frame of interest to a reference frame computes a transform between the frame of interest and a selected key frame from a set of key frames 308 and accumulates that transform with a transform from the selected key frame to the reference frame. For example, homographic transforms may be used, as follows:

$${H}_{i \to o} = {H}_{k \to o} * {H}_{i \to k}$$

where, i denotes the frame of interest, o denotes the reference frame, and k denotes the selected key frame. In the illustrated example, a homographic transform is computed as follows:

$${H}_{3 \to 0} = {H}_{k \to 0} * {H}_{3 \to k}$$

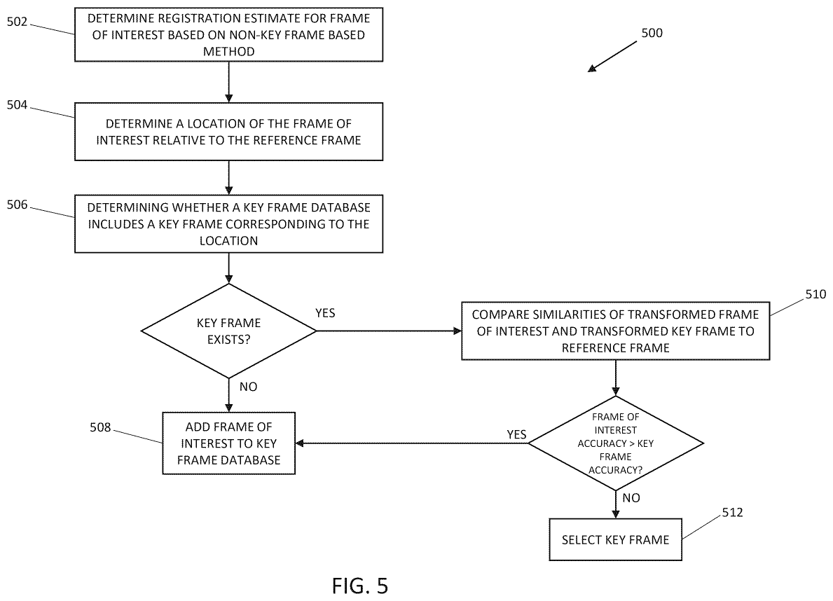

The set of key frames may include frames of the series of frames that were captured between when the reference frame was captured and when the frame of interest was captured. A key frame from the set of key frames 308 for use in computing the transform according to registration method 306 may be selected based on a similarity between the frame of interest and the key frame. Examples for determining a similarity between the frame of interest and the key frame are described below with reference to FIG. 5.

Although the above example shows a single key frame being used to determine the registration estimate for the frame of interest, other examples include using more than one key frame. For example, a first key frame may be selected that is similar to the frame of interest and a transform for that first key frame to a second key frame in the key frame data base may be accumulated with a transform from the second key frame to the reference frame (directly or via any number of additional key frames) and a transform from the frame of interest to the first key frame.

Key frame matching according to registration method 306 can improve registration accuracy by avoiding the divergency that may occur with the adjacent transform accumulation performed, for example, in first registration method 302. This may be particularly true when the frame of interest has limited overlap with the reference frame (e.g., due to camera motion and/or self-motion from targets). The key frame matching approach of registration method 306 can provide an optimal intermediate transform transition for the frame of interest back to the reference frame.

As noted above, a transform that may be determined according to any of the above registration methods is a homographic transform. Any method of estimating a homographic transform between two images can be used. Optionally, a homographic transform is estimated by a random sample consensus (RANSAC) algorithm. The RANSAC algorithm may estimate the homography based on matched features in the two images. The matched features may be determined by a suitable feature detector, such as the Scale-Invariant Feature Transform (SIFT) algorithm and a matcher, such as the K-nearest-neighborhood (KNN) matcher. In general, the RANSAC algorithm performs multiple iterations of selecting a subset of the matched features, computing a homography from that set of matched features, and determining the number of other matched features that are consistent with that homography (determining the inliers). The number of iterations performed is tunable and can be, for example, hundreds of iterations (e.g., a thousand iterations), each using a different subset of matched features. The homography that has the highest inlier ratio (the ratio of the total number of inliers for that homography to the total number of matched features) is provided as the homography for the two images. Other similar homography estimators that may be used include N adjacent points sample consensus (NAPSAC), progressive sample consensus (PROSAC), marginalizing sample consensus (MAGSAC), and MAGSAC++. Machine learning-based local feature finders (e.g., GLAMPoints) and machine learning-based point-matchers (e.g., SuperGlue) may also be used to estimate the homography between two images. Other transform types that may be used include affine or translation transforms, piecewise affine transforms, or dense motion vector field transforms.

Returning to FIG. 2, at step 206, a registration accuracy may be determined for one or more registration estimates determined in step 204. For example, a registration accuracy may be determined for a registration estimate determined based on registration method 302, a registration accuracy may be determined for a registration estimate determined based on registration method 304, and/or a registration accuracy may be determined for a registration estimate determined based on registration method 306.

An example of a registration accuracy that may be used is an inlier ratio R determined by, for example, the RANSAC algorithm and other consensus methods that may be used to estimate the homography, as described above. The inlier ratio R can be defined as the ratio of the number of matched features that fit the homography H determined by the RANSAC algorithm to the total number of matched features, as follows:

$$R = \frac{number of matched features used to estimate H}{total number of matched features}$$

The greater the value of R, the greater the registration accuracy. Where the registration method accumulates multiple transforms, such as method 302 and/or method 306 of FIG. 3, the registration accuracy for the resulting registration estimate may be the inlier ratio of the transform from the frame of interest to an adjacent frame in the accumulation (e.g., from the frame of interest to the adjacent frame in the series in the case of method 302 and from the frame of interest to the key frame in the case of method 306).

Another example of a registration accuracy that may be used is key point reprojection error. Calculating the key point reprojection error may include measuring the mean square error between locations of the key points from the reference frame and the locations of transformed key points (key points warped by the homography transform) from the frame of interest.

Other registration accuracy metrics may be used, including computing a similarity metric (e.g., structural similarity index measure (SSIM)) between the reference frame and a frame of interest that has been warped according to the determined transform. Another example of a registration accuracy metric is the detected key points reprojection error.

Although step 206 is illustrated in the flow diagram of FIG. 2 as a subsequent step to step 204, this is merely for illustrating that registration accuracies are determined for the registration estimates and does not indicate that the registration accuracy must be determined after the registration estimate (though it may be in some variations). As noted above, a registration accuracy, such as the inlier ratio, may be generated in the process of generating the registration estimate.

At step 208, one of the registration estimates determined in step 204 is selected based on the registration accuracies determined at step 206. For example, where a registration estimate determined using registration method 304 has a registration accuracy that is greater than a registration accuracy of a registration estimate determined using registration method 302, the registration estimate determined using registration method 304 may be selected in step 208.

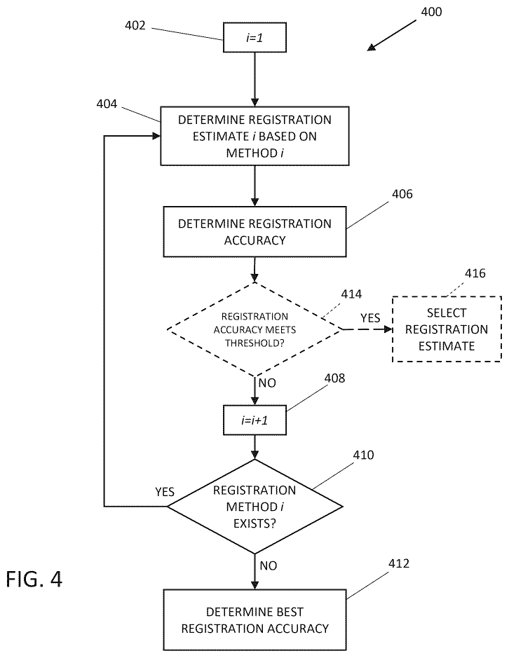

Optionally, registration estimates are determined for multiple available registration methods (e.g., for each available registration method) and the registration accuracies are then compared to determined which registration estimate to select. An example of this approach to performing steps 204 to 208 is illustrated by the exemplary flow diagram of FIG. 4. Method 400 of FIG. 4 includes setting a counter i to 1 at step 402. At step 404, a registration estimate i is determined based on registration method i. At step 406, a registration accuracy of registration estimate i is determined. At step 408, the counter i is incremented by 1. At step 410, a determination is made whether registration method i exists (i.e., whether there is another registration method that has not been used). If it does, then method 400 returns to step 404 for determining a registration estimate using the next registration method. If all registration methods have been used (i.e., “no” at step 410), then at step 412, a determination is made of which registration estimate has the best registration accuracy and the registration estimate with the best registration accuracy can be selected according to step 208 of method 200. Thus, method 400 is an exemplary method for performing steps 204 to 208 of method 200.

Optionally, registration estimates are determined until a registration estimate has a registration accuracy that meets a threshold. For example, an inlier ratio threshold of 80% may be set and one or more registration estimates may be determined according to different registration methods until a registration estimate has an inlier ratio that equals or exceeds 80%, which is the selected registration estimate according to step 208. As such, whether a registration estimate is determined according to a given registration method may be predicated on whether a previously determined registration estimate (determined according to a different registration method) meets the predetermined threshold. An example of this approach for selecting a registration estimate is illustrated by optional steps 414 and 416 in the flow diagram of FIG. 4. At step 414, the registration accuracy determined in step 406 is compared to a threshold. If it does not meet the threshold, then method 500 continues to step 408. However, if the threshold is met in step 414, then at step 416, the registration estimate is selected, and no further registration estimates are determined. Each registration estimate may be compared to the threshold in this way until a registration estimate meets the registration accuracy threshold. If none do, then after all registration methods have been used, the registration estimate with the best accuracy is determined in step 412. The iterative approach introduced by optional steps 414 and 416 can lead to lower computation time compared to an approach in which every registration method is used every time. Optionally, the iterative approach can follow a predefined order of registration methods. For example, with reference to the registration methods of FIG. 3, registration method 302 may be used first and its registration accuracy compared to a threshold. If it does not meet the threshold, then registration method 306 may be used next. If its registration accuracy also fails to meet the threshold, then registration method 304 may be used. This is just one example of an order of registration methods that may be used, and it should be understood that any order of the registration methods of FIG. 3 with or without any other registration methods may be used.

Returning to FIG. 2, at step 210, the selected registration estimate is stored in memory as the registration of the frame of interest to the reference frame. The registration for the frame of interest to the reference frame can be used to register fluorescence light frames that are captured close in time to the frame of interest. For example, a fluorescence light frame that is captured close in time to the frame of interest can be registered to a fluorescence light frame captured close in time to the reference frame using the stored registration. Other uses of the registration stored at step 210 include motion deblurring, virtual marker placement and visualization, and navigation. The registration of the frame of interest to the reference frame may be used for registering one or more subsequently captured frames to the reference frame. For example, a transform from a subsequently captured frame back to the reference frame may accumulate a transform from the subsequently captured frame to the frame interest and the stored registration for the frame of interest.

Optionally, step 210 can include storing a transform from the frame of interest to a prior frame, such as a previously captured frame, which can be used during registration of a subsequently captured frame. For example, the transform (e.g., H_{n→n-1}) from frame of interest fₙ to previous frame fₙ₋₁ can be stored and used for determining a registration estimate for a next frame fₙ₊₁ according to method 302 by including the transform (H_{n→n-1}) in the accumulation of transforms back to the reference frame.

As noted above, a registration method (e.g., registration method 306 of FIG. 3) may include selecting a key frame from a set of key frames based on a similarity between the frame of interest and the selected key frame. FIG. 5 is a flow diagram of an exemplary method 500 for determining which key frame from among a set of key frames to select for the frame of interest and for adding frames to the set of key frames. Method 500 uses locations of the frame of interest and key frames relative to a reference frame as a proxy for similarity between the frame of interest and the key frames. Method 500 may be performed for steps of method 200 of FIG. 2, such as for steps 204 and 206 of method 200.

At step 502, a registration estimate for registering the frame of interest to the reference frame is determined using a registration method that does not use key frames. The registration method can be, for example, registration method 302 or registration method 304 of FIG. 3.

At step 504, a location of the frame of interest relative to the reference frame is determined based on the registration estimate from step 502. The location of the frame of interest relative to the reference frame may be a location of a center of the frame of interest relative to a center of the reference frame. The location of a center of the frame of interest relative to the center of the reference frame can be determined by applying the transform from the registration estimate to the center of the frame of interest and determining the difference in location of the center of the frame of interest (registered to the reference frame) to the center of the reference frame. The difference in location can be, for example, a distance and direction of the center of the frame of interest to the reference frame in the x- and y-dimensions of the reference frame. Distances can be in pixels or can be in another unit of measurement, such as millimeters.

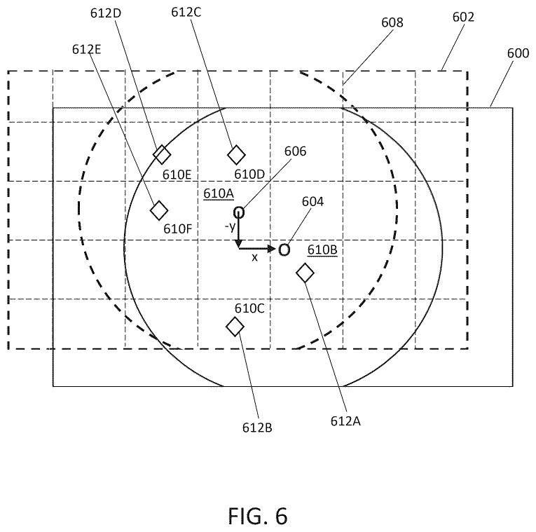

FIG. 6 illustrates an example of determining the location of a center of a frame of interest relative to a center of a reference frame. In the illustrated example, the frame of interest 600 is shown shifted relative to the reference frame 602 according to the registration estimate determined at step 502. For example, assuming the frame of interest 600 was captured by a camera that moved relative to its position when it captured the reference frame 602 by a simple translation of the equivalent of 50 pixels in the x-direction of the reference frame and 50 pixels in the negative y-direction of the reference frame 602, the registration estimate determined at step 502 may be a transform that shifts pixels of the frame of interest 600 by an amount of 50 pixels in the negative x-direction and an amount of 50 pixels in the y-direction to register to the reference frame 602. Thus, in applying the transform to the frame of interest 600, the center 604 of the frame of interest 600 is shifted in the x-direction by 50 pixels and in the negative y-direction by 50 pixels from the center 606 of the reference frame 602.

At step 506, a determination is made whether there is a key frame stored in a key frame database that has a location that corresponds to the location of the frame of interest determined in step 504. The location of the key frame need not be the exact location of the frame of interest. Rather, the key frame may be within a threshold distance of the location of the frame of interest or may be within a predefined region of locations that also encompasses the frame of interest.

FIG. 6 illustrates an example of the logical organization of a key frame database in a hash table that uses regions associated with the reference frame. A hash table is constructed based on a grid 608 that is defined for the reference frame 602. Each grid cell (e.g., cells 610A, 610B) defines a region of locations relative to the center 606 of the reference frame. One or more of these cells can be assigned a corresponding a single key frame based on the center of that key frame falling within the region associated with the cell. So, for example, if the frame of interest 600 were a key frame, it may be assigned to cell 610B since its center 604 falls within cell 610B. In the illustrated example, key frames 612A, B, C, D, and E are assigned to cells 610B, C, D, E, and F, respectively.

The size of the grid (e.g., its x- and y-dimensions) may be equal to the size of the reference frame 602 or can be bigger or smaller that the reference frame 602. The number of cells that the grid has may be selected based on various considerations such as registration accuracy, memory space, and key frame management computing resource requirements, with a denser grid being associated with improved registration accuracy, greater memory space requirements, and greater key frame management computing resource requirements. Each cell may be defined in the key frame database by its x- and y-extends. For example, cell 610A may be defined as extending from −40 pixels to +40 pixels in the x-direction and from −40 pixels to +40 pixels in the y-direction relative to the center 606 of the reference frame 602 and cell 610B may be defined as extending from +40 pixels in the x-direction to +80 pixels in the x-direction and from −40 pixels in the y-direction to −80 pixels in the y-direction. The key frame database can include an entry for each cell, the entry defining the location and extent of the cell and, if a key frame has been assigned to it, an identifier for the key frame (so that the key frame can be located in memory), and a registration for the key frame back to the reference frame.

The two-dimensional grid approach described above is merely one example of a way of organizing a key frame database. Key frames may be defined using more complex spatial hash maps, which may include one or more additional dimensions, such as zoom as a third dimension. Another suitable approach to organizing a key frame data base is to use a nearest neighbor spatial hash-map lookup (e.g., using octree), which may improve reliability and accuracy without imposing substantial additional computational costs.

Returning to step 506, the grid 608 may be used to determine whether there is a key frame in the key frame database that corresponds to the location of the frame of interest 600 by determining which cell the center 604 of the frame of interest 600 falls within. For example, in the illustrated example, the center 604 of the frame of interest 600 falls within cell 610B, so step 506 can include determining whether a key frame is assigned to cell 610B. In the illustrated example, key frame 612A is assigned to cell 610B.

If there is no key frame in the key frame database that corresponds to the location of the frame of interest, then the frame of interest may be added to the key frame database at step 508. For example, if key frame 612A had not been assigned to cell 610B, then the frame of interest 600 can be added to the key frame database and assigned to cell 610B. In this way, the key frame database can be built up over time as new frames are captured and registered to the references frame. The farther the camera moves from its reference frame position, the more key frames can be added to the key frame database.

If the key frame database does include a key frame that corresponds to the location of the frame of interest relative to the reference frame, then at step 510, a similarity of the key frame registered to the reference frame and a similarity of the frame of interest registered to the reference frame are compared. This may include comparing a registration accuracy of the registration estimate for the frame of interest (as determined in step 502) to a registration accuracy of the key frame (the registration accuracy of the key frame may be stored in the key frame database in association with the key frame). If the registration accuracy of the registration estimate for the frame of interest is greater than the key frame registration accuracy (or greater than the key frame registration accuracy by a threshold amount), then at step 508, the frame of interest is added to the key frame data base by replacing the key frame. If the registration accuracy of the registration estimate for the frame of interest is less than the key frame registration accuracy (or less than a threshold amount greater than the key frame registration accuracy), then at step 512, the key frame is selected for use in determining the registration estimate for the frame of interest according to method 306 of FIG. 3. By comparing the registration accuracies of new frames to the registration accuracies of the key frames in the key frame database, the key frame database can be improved over time. Optionally, step 510 is not performed and, instead, the key frame is selected in step 512.

Efficient key frame management, as described above, can enable a key frame-based registration approach executable with low latency. The two-dimensional hash table approach described above has O(1) search complexity regardless of the number of key frames. This can increase the efficiency of key frame matching with low latency.

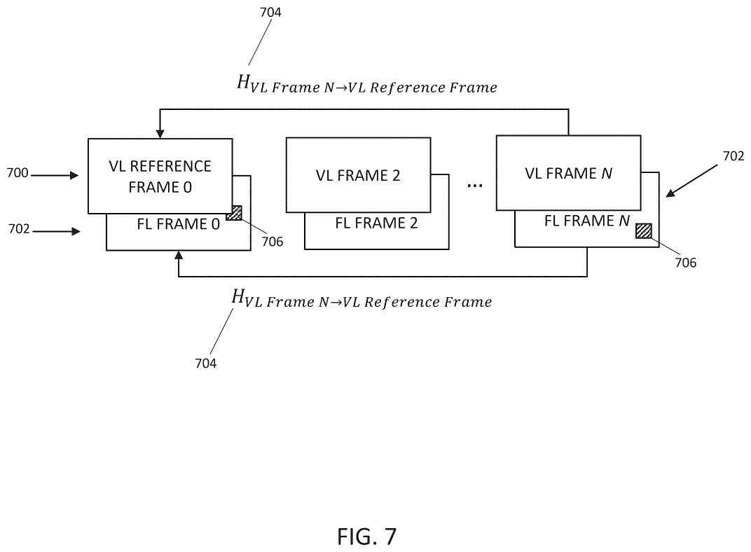

Returning to method 200 of FIG. 2, the registration for the frame of interest stored in step 210 can be used in a number of different ways. In general, the registration can be used to track temporal information associated with the sequence of frames or an associated sequence of frames (e.g., a fluorescence signal associated with fluorescence frames captured simultaneously with visible light frames) and determine its dynamic changes in the time domain. For example, as illustrated in FIG. 7, registration of a series of visible light frames 700 can be used to register pixel data of a series of fluorescence light frames 702 enabling comparison between the fluorescence signal associated with the same portion of the imaged scene across the series of fluorescence light frames 702 in the presence of motion of the camera and/or the scene. In FIG. 7, fluorescence frames and visible light frames are captured in alternating fashion. A registration 704 is determined for registering a visible light frame N back to the visible light reference frame 0. This same registration 704 is then used to register fluorescence frame N to fluorescence frame 0. With the fluorescence frames registered to each other, a comparison between fluorescence response for the same location 706 can be made despite the location having moved within the field of view over time. This could be used, for example, to calculate characteristics of fluorescence intensity over time for each region of imaged tissue. Such characteristics could be represented as a color overlay that may be warped to fit a frame of interest using the registration estimates. The overlay can be merged with the visible light frame and displayed to a user.

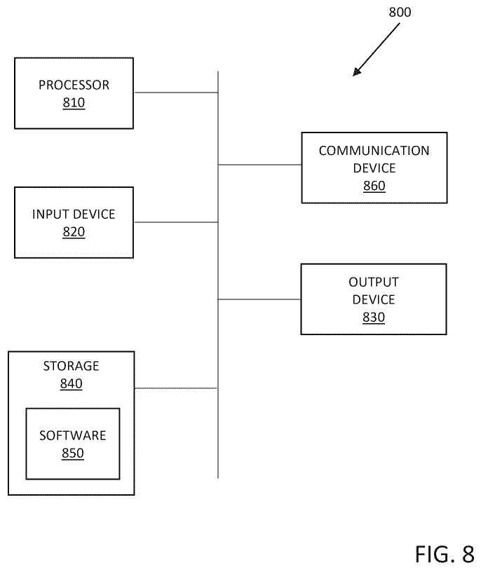

FIG. 8 illustrates an example of a computing system 800 that can be used for one or more components of system 100 of FIG. 1, such as one or more of light source 108, camera control unit 120, imager 104, and image processing unit 122. System 800 can be a computer connected to a network, such as one or more networks of hospital, including a local area network within a room of a medical facility and a network linking different portions of the medical facility. System 800 can be a client or a server. System 800 can be any suitable type of processor-based system, such as a personal computer, workstation, server, handheld computing device (portable electronic device) such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), graphics processing unit (GPU), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices as described above), such as programming for performing one or more steps of method 200 of FIG. 2, method 400 of FIG. 4, and/or method 500 of FIG. 5.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer readable medium can include, but is not limited to, an electronic, magnetic, optical, electromagnetic, or infrared wired or wireless propagation medium.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method of registering frames of a medical video comprising, at a computing system:
receiving a series of video frames captured by a medical imager imaging a scene of interest of a patient;
for a first frame of the series of video frames, determining a plurality of registration estimates for registering the first frame to a second frame of the series of video frames, wherein each registration estimate is determined according to a different one of a plurality of registration methods;
determining registration accuracies for the plurality of registration estimates;
selecting one of the plurality of registration estimates based on the registration accuracies; and
storing in a memory the selected registration estimate as a registration of the first frame to the second frame.

2. The method of claim 1, wherein the series of video frames comprises at least one frame that was captured between the first frame and the second frame, and the plurality of registration methods comprises at least one registration method that includes calculating a transform from the first frame directly to the second frame.

3. The method of claim 1, wherein the plurality of registration methods comprises at least one registration method that comprises calculating a transform from the first frame to an intermediate frame that was captured between the first and second frames.

4. The method of claim 3, wherein the at least one registration method comprises combining the transform from the first frame to the intermediate frame with a plurality of transforms associated with frames captured between the intermediate frame and the second frame.

5. The method of claim 3, wherein the intermediate frame is a key frame and the at least one registration method comprises combining the transform from the first frame to the key frame with a second transform from the key frame to the second frame.

6. The method of claim 5, wherein the key frame is a selected key frame that was selected from a plurality of key frames based on a similarity between the first frame and the key frame.

7. The method of claim 6, wherein the at least one registration method that comprises combining the transform from the first frame to the selected key frame with the second transform is a first registration method and the selected key frame is selected based on a second registration method.

8. The method of claim 7, wherein the second registration method comprises combining a plurality of transforms associated with frames captured between the first frame and the second frame.

9. The method of claim 7, comprising determining a relative difference in location between the first frame and the second frame, wherein the selected key frame is selected based on the relative difference in location between the first frame and the second frame.

10. The method of claim 9, wherein the relative difference in location between the first frame and the second frame comprises a relative difference in location between a center of the first frame and a center of the second frame.

11. The method of claim 9, wherein the selected key frame is selected by determining which key frame of the plurality of key frames has a location corresponding to the first frame.

12. The method of claim 11, wherein each key frame of the plurality of key frames is associated with a range of locations relative to the second frame and the selected key frame is selected by determining that the relative difference in location between the first frame and the second frame is within a range associated with the selected key frame.

13. The method of claim 6, wherein the plurality of key frames are frames of the series of video frames.

14. The method of claim 1 comprising:
determining a location of the first frame relative to the second frame based on a first registration estimate;
determining whether a key frame database includes a key frame that is sufficiently close in location to the first frame;
in accordance with the key frame database not including a key frame that is sufficiently close in location to the first frame, adding the first frame to the key frame database; and
in accordance with the key frame database including a key frame that is sufficiently close in location to the first frame: comparing a registration accuracy associated with the key frame and a registration accuracy associated with the first registration estimate for the first frame, and replacing the key frame with the first frame if the registration accuracy associated with the first registration estimate is greater than the registration accuracy associated with the key frame.

15. The method of claim 1, wherein selecting the one of the plurality of registration estimates based on the registration accuracies comprises comparing a first registration accuracy corresponding to a first registration estimate to a threshold, and in accordance with the first registration accuracy not meeting the threshold, comparing the first registration accuracy to a second registration accuracy corresponding to a second registration estimate.

16. The method of claim 1, wherein at least one registration method of the plurality of registration methods comprises determining at least one homography estimate.

17. The method of claim 16, wherein the at least one homography estimate is determined using a random sample consensus algorithm.

18. The method of claim 16, wherein the at least one homography estimate is determined using a machine learning-based algorithm.

19. The method of claim 1, wherein the medical imager is an endoscopic imager.

20. The method of claim 1 comprising registering pixel data of the first frame to pixel data of the second frame based on the selected registration estimate.

21. The method of claim 1, wherein the series of video frames is associated with a first imaging modality, and the method comprises registering a frame of a series of video frames associated with a second imaging modality based on the selected registration estimate.

22. A system for registering frames of a medical video comprising one or more processors and memory storing one or more programs for execution by the one or more processors, the one or more programs including instructions that when executed by the one or more processors cause the system to perform a method comprising:
receiving a series of video frames captured by a medical imager imaging a scene of interest of a patient;
for a first frame of the series of video frames, determining a plurality of registration estimates for registering the first frame to a second frame of the series of video frames, wherein each registration estimate is determined according to a different one of a plurality of registration methods;
determining registration accuracies for the plurality of registration estimates;
selecting one of the plurality of registration estimates based on the registration accuracies; and
storing in the memory the selected registration estimate as a registration of the first frame to the second frame.

23. The system of claim 22 comprising the medical imager.

24. A non-transitory computer readable storage medium storing one or more programs for execution by one or more processors of a computing system, the one or more programs including instructions for causing the computing system to perform a method comprising:
receiving a series of video frames captured by a medical imager imaging a scene of interest of a patient;
for a first frame of the series of video frames, determining a plurality of registration estimates for registering the first frame to a second frame of the series of video frames, wherein each registration estimate is determined according to a different one of a plurality of registration methods;
determining registration accuracies for the plurality of registration estimates;
selecting one of the plurality of registration estimates based on the registration accuracies; and
storing in the memory the selected registration estimate as a registration of the first frame to the second frame.
